(19) United States
(12) Patent Application Publication
(10) Pub. No.: US 2023/0207078 A1
(43) Pub. Date: Jun. 29, 2023

(54) SYSTEMS AND METHODS FOR REMOTE DIAGNOSTIC TESTING AND TREATMENT

(71) Applicant: EMED LABS, LLC, Miami, FL (US)

(72) Inventors: Zachary Carl Nienstedt, Wilton Manors, FL (US); Nicholas Atkinson Kramer, Seattle, WA (US); Adam Charles Carlson, Miami, FL (US); Colman Thomas Bryant, Fort Lauderdale, FL (US); Igor Javier Rodriguez, Miami, FL (US); Marco Magistri, Miami, FL (US)

(21) Appl. No.: 18/065,913

(22) Filed: Dec. 14, 2022

Related U.S. Application Data
(60) Provisional application No. 63/266,224, filed on Dec. 30, 2021.
(60) Provisional application No. 63/266,211, filed on Dec. 30, 2021.
(60) Provisional application No. 63/266,233, filed on Dec. 30, 2021.
(60) Provisional application No. 63/266,158, filed on Dec. 29, 2021.
(60) Provisional application No. 63/294,702, filed on Dec. 29, 2021.
(60) Provisional application No. 63/294,731, filed on Dec. 29, 2021.
(60) Provisional application No. 63/266,160, filed on Dec. 29, 2021.
(60) Provisional application No. 63/299,048, filed on Jan. 13, 2022.

(51) Int. Cl. G16H 10/60; G16H 40/67

(52) U.S. Cl. CPC G16H 10/60; G16H 40/67

(57) ABSTRACT

A method or system can include receiving patient information from a patient that includes a result of at least one remotely-administered diagnostic test, wherein the at least one remotely-administered diagnostic test, generating, a nutritional profile for the patient based on the patient information, transmitting the nutritional profile to one or more food-related services, wherein the food-related services provide a service to the user based at least in part on the nutritional profile, receiving information regarding an interaction between the patient and at least one of the food-related services, and updating the nutritional profile of the patient based on the information received from the at least one of the food-related services

### PRIORITY APPLICATIONS

This application claims priority to: (1) U.S. Provisional Patent Application No. 63/290,482, filed Dec. 16, 2021, (2) U.S. Provisional Patent Application No. 63/266,160, filed Dec. 29, 2021, (3) U.S. Provisional Patent Application No. 63/294,731, filed Dec. 29, 2021, (4) U.S. Provisional Patent Application No. 63/294,702, filed Dec. 29, 2021, (5) U.S. Provisional Patent Application No. 63/266,158, filed Dec. 29, 2021, (6) U.S. Provisional Patent Application No. 63/266,233, filed Dec. 30, 2021, (7) U.S. Provisional Patent Application No. 63/266,211, filed Dec. 30, 2021, (8) U.S. Provisional Patent Application No. 63/266,224, filed Dec. 30, 2021, each of which are incorporated herein by reference in their entirety. Any and all applications for which a foreign or domestic priority claim is identified in the Application Data Sheet as filed with the present application are hereby incorporated by reference under 37 CFR 1.57.

### BACKGROUND

### Field

The present application is directed to systems, methods, and devices for remote diagnostic testing and treatment.

### Description

Use of telehealth to deliver healthcare services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

In a first aspect, a computer-implemented method is disclosed which includes: receiving, by a computer system, patient information from a patient in communication with the computer system over an electronic network, the patient information comprising a result of at least one remotely-administered diagnostic test, wherein the at least one remotely-administered diagnostic test is facilitated through an testing platform accessed by the patient over the electronic network; generating, by the computer system, a nutritional profile for the patient based on the patient information; transmitting, by the computer system, the nutritional profile over the electronic network to one or more food-related services, wherein the food-related services provide a service to the user based at least in part on the nutritional profile; receiving, by the computer system, information regarding an interaction between the patient and at least one of the food-related services, wherein the information is sent by the food-related services over the electronic network to the computer system; and updating, by the computer system, the nutritional profile of the patient based on the information received from the at least one of the food-related services.

In some embodiments the patient information comprises one or more of: a white blood cell count of the patient; an indicator of kidney function for the patient; an indicator of liver function for the patient; an indication of a hydration level for the patient; an indicator of a pH level of the patient; an indicator of a measure of ketones of the patient; and/or allergy information for the patient.

In some embodiments, the method further includes receiving, by a computer system, additional patient information from a healthcare provider or medical device in communication with the computer system over an electronic network, the patient information comprising a result of at least one remotely-administered diagnostic test, wherein the at least one remotely-administered diagnostic test is facilitated through an testing platform accessed by the patient over the electronic network. In some embodiments, the additional information comprises one or more of: a weight of the patient; a blood pressure of the patient; a glucose level of the patient; a cholesterol measure of the patient; a medical condition of the patient; a medications taken by the patient; dietary habits and/or preferences of the patient; a wellness goal of the patient; and/or a biomarker metric of the patient.

In some embodiments, the nutritional profile is further based on the additional information. In some embodiments, the food-related services comprises one or more of the following: a rapid food delivery service; a grocery ordering and/or delivery service; a subscription meal services; a restaurant; and/or an establishment dining service.

In another aspect, a system can include at least one memory storing instructions that cause at least one processor to: receive patient information from a patient in communication with the computer system over an electronic network, the patient information comprising a result of at least one remotely-administered diagnostic test, wherein the at least one remotely-administered diagnostic test is facilitated through an testing platform accessed by the patient over the electronic network; generate a nutritional profile for the patient based on the patient information; transmit the nutritional profile over the electronic network to one or more food-related services, wherein the food-related services provide a service to the user based at least in part on the nutritional profile; receive information regarding an interaction between the patient and at least one of the food-related services, wherein the information is sent by the food-related services over the electronic network to the computer system; and update the nutritional profile of the patient based on the information received from the at least one of the food-related services.

In some embodiments, the patient information comprises one or more of: a white blood cell count of the patient; an indicator of kidney function for the patient; an indicator of liver function for the patient; an indication of a hydration level for the patient; an indicator of a pH level of the patient; an indicator of a measure of ketones of the patient; and/or allergy information for the patient.

In some embodiments, the instructions further cause the processor to receive additional patient information from a healthcare provider or medical device in communication with the computer system over an electronic network, the patient information comprising a result of at least one remotely-administered diagnostic test, wherein the at least one remotely-administered diagnostic test is facilitated through an testing platform accessed by the patient over the electronic network. In some embodiments, the additional information comprises one or more of: a weight of the patient; a blood pressure of the patient; a glucose level of the patient; a cholesterol measure of the patient; a medical condition of the patient; a medications taken by the patient; dietary habits and/or preferences of the patient; a wellness goal of the patient; and/or a biomarker metric of the patient.

In some embodiments, the nutritional profile is further based on the additional information. In some embodiments, the food-related services comprises one or more of the following: a rapid food delivery service; a grocery ordering and/or delivery service; a subscription meal services; a restaurant; and/or an establishment dining service.

In another aspect, a computer-implemented method is disclosed which includes determining, by a computer system, a test result of a user, wherein the test result based on a self-administered diagnostic medical test; based upon a determination that the test result is positive, wherein a positive test result indicates that treatment is advisable: transmitting, by the computer system, a prescription questionnaire to the user, the prescription questionnaire, wherein the prescription questionnaire is transmitted to the user over an electronic network and displayed to the user on a user device; receiving, by the computer system, user inputs in response to the prescription questionnaire, wherein the user inputs are inputted by the user on the user device and transmitted to the computer system over the electronic network; transmitting, by the computer system, the user inputs and the test result to a prescription provider, wherein the user inputs are transmitted over the electronic network and displayed the prescription provider on a provider device, and wherein the prescription provider determines at least one prescription for the user based on the user inputs and the test result; receiving, by the computer system, prescription information based on the at least one prescription determined by the prescription provider, wherein the prescription information is transmitted to the computer system over the electronic network; determining, by the computer system, a delivery partner based on a location of the user and a location of the prescription provider, a delivery partner for delivering the prescription to the user; generating, by the computer system, delivery instructions for the delivery partner, wherein the delivery instructions are configured to facilitate the delivery partner obtaining the prescription from the prescription provider and delivering the prescription to the user; and transmitting, by the computer system, the delivery instructions the delivery partner, wherein the delivery instructions are transmitted over the electronic network.

In some embodiments, the self-administered medical diagnostic test is taken by the user using a testing platform provided by the computer system that provides a video conference connection between the user and a proctor.

In some embodiments, the method further includes determining, by the computer system, the prescription questionnaire based on the test result and a database of available medications.

In some embodiments, the prescription questionnaire is further based on a user profile, wherein the user profile is generated based on an interaction between the user and using a testing platform provided by the computer system that facilitates the self-administered medical diagnostic test.

In some embodiments, the method further includes determining, by the computer system, if more than a threshold period of time has passed since the user obtained the valid positive test result.

In another aspect, a system can include at least one memory storing instructions that cause the at least one processor to: determine a test result of a user, wherein the test result based on a self-administered diagnostic medical test; based upon a determination that the test result is positive, wherein a positive test result indicates that treatment is advisable: transmit a prescription questionnaire to the user, the prescription questionnaire, wherein the prescription questionnaire is transmitted to the user over an electronic network and displayed to the user on a user device; receive user inputs in response to the prescription questionnaire, wherein the user inputs are inputted by the user on the user device and transmitted to the computer system over the electronic network; transmit the user inputs and the test result to a prescription provider, wherein the user inputs are transmitted over the electronic network and displayed the prescription provider on a provider device, and wherein the prescription provider determines at least one prescription for the user based on the user inputs and the test result; receive prescription information based on the at least one prescription determined by the prescription provider, wherein the prescription information is transmitted to the computer system over the electronic network; determine a delivery partner based on a location of the user and a location of the prescription provider, a delivery partner for delivering the prescription to the user; generate delivery instructions for the delivery partner, wherein the delivery instructions are configured to facilitate the delivery partner obtaining the prescription from the prescription provider and delivering the prescription to the user; transmit the delivery instructions the delivery partner, wherein the delivery instructions are transmitted over the electronic network.

In some embodiments, the self-administered medical diagnostic test is taken by the user using a testing platform provided by the computer system that provides a video conference connection between the user and a proctor.

In some embodiments, the instructions further cause the processor to determine the prescription questionnaire based on the test result and a database of available medications.

In some embodiments, the prescription questionnaire is further based on a user profile, wherein the user profile is generated based on an interaction between the user and using a testing platform provided by the computer system that facilitates the self-administered medical diagnostic test.

In some embodiments, the instructions further cause the processor to determine if more than a threshold period of time has passed since the user obtained the valid positive test result.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present application are described with reference to drawings of certain embodiments, which are intended to illustrate, but not limit the present disclosure. It is to be understood that the attached drawings are for the purpose of illustrating concepts disclosed in the present application and may not be to scale;

FIG. 1 is a block diagram illustrating an embodiment of a protocol or method for personalizing food-related services based on health or wellness data.

FIG. 2 is a block diagram illustrating an embodiment of a protocol or method for an example tabletop accessory.

FIG. 3 is a block diagram illustrating an embodiment of another protocol or method for an example tabletop accessory.

FIG. 4 is a block diagram illustrating testing and treatment according to some embodiments.

FIG. 5 is a block diagram illustrating an embodiment of a protocol or method for determining a treatment to order.

FIG. 6 is a block diagram an embodiment of a protocol or method for testing and treatment determination.

FIG. 7 is a flow chart illustrating a sample user experience for receiving remote treatment according to some embodiments.

FIG. 8 shows an illustration of an embodiment of a method or protocol for professionally-administered treatment.

FIG. 9 shows an illustration of another embodiment of a method or protocol for professionally-administered treatment.

FIG. 10 illustrates an embodiment of a test cards for diagnostic testing.

FIG. 11 illustrates another embodiment of a test cards for diagnostic testing.

FIG. 12 illustrates another embodiment of a test cards for diagnostic testing.

FIG. 13 illustrates another embodiment of a test cards for diagnostic testing.

FIG. 14 illustrates an embodiment of a test reader device for diagnostic testing

FIG. 15 illustrates another embodiment of a test cards for diagnostic testing.

FIG. 16 is a block diagram illustrating an embodiment of a computer hardware system configured to run software for implementing one or more embodiments of the health testing and diagnostic systems, methods, and devices disclosed herein.

### DETAILED DESCRIPTION

Although several embodiments, examples and illustrations are disclosed below, it will be understood by those of ordinary skill in the art that inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

### Personalized Food Services Based on Nutritional Profile

Data related to a user's health and/or wellness can be gathered from a variety of places, including diagnostic health tests, health-related wearables (e.g., fitness or other health trackers, user input, etc.). As described herein, a user's health or wellness data may be used to generate a nutritional profile specific to the user. In some instances, the nutritional profile may be used to personalize the experience of at least one food-related service for the user.

In some embodiments, the user may provide health or wellness data to the platform. Data can be provided in various ways, including data input by the user or a healthcare professional, data obtained from one or more diagnostic health tests, and/or data provided by one or more health-related wearable devices. This may allow the platform to generate the nutritional profile for the user based on the data provided. The platform may then share the user's nutritional profile with food-related services, which can allow the food-related service to utilize the user's nutritional profile to provide the user with a more personalized experience. Additionally, information about the various orders the user may place through the food-related services may be fed back to the platform. The platform may then update the user's nutritional profile. This can, for example, beneficially assist a user with specific dietary preferences and/or restrictions in navigating food-related services.

In some instances, a user may provide health or wellness data to a platform. Such health or wellness data may include results from diagnostic tests or data provided by the user, a wellness professional, a health or medical device, and/or one or more services connected with the platform, or the like. Results from a diagnostic test may include, but are not limited to, white blood cell count, kidney function, liver function, hydration levels, pH levels, urinary tract infection, ketones, allergies, etc. The data provided may include information such as the user's weight, blood pressure, glucose levels, cholesterol, medical conditions, medications or over-the-counter products taken by the user, dietary habits and/or preferences, wellness goals, biomarker metrics, etc. Subsequently, the platform may generate and/or update a nutritional profile designated to the user based on the wellness data provided. The platform may share the user's nutritional profile with at least one food-related service. Such food-related services may include rapid food delivery services, grocery ordering or delivery services, subscription meal services, independent restaurants or restaurant chains, establishment dining services, etc. The at least one food-related service may then utilize the user's nutritional profile to provide the user with a more personalized experience. Subsequently, the information regarding the orders the user placed through the one or more food-related services may be fed back to the platform and used to update the user's nutritional profile.

Navigating food-related services to find products that fit the user's dietary needs, preferences, or restrictions may be difficult. Accordingly, it may be beneficial to provide the user with personalized food-related services. This may allow the user to make use of various food-related services more easily. For example, in some cases, personalization can be based on health or wellness data collected from various diagnostic tests or other health or wellness data provided. Furthermore, for example, food-related services may personalize a menu or list of food-related items based on the user's wellness data.

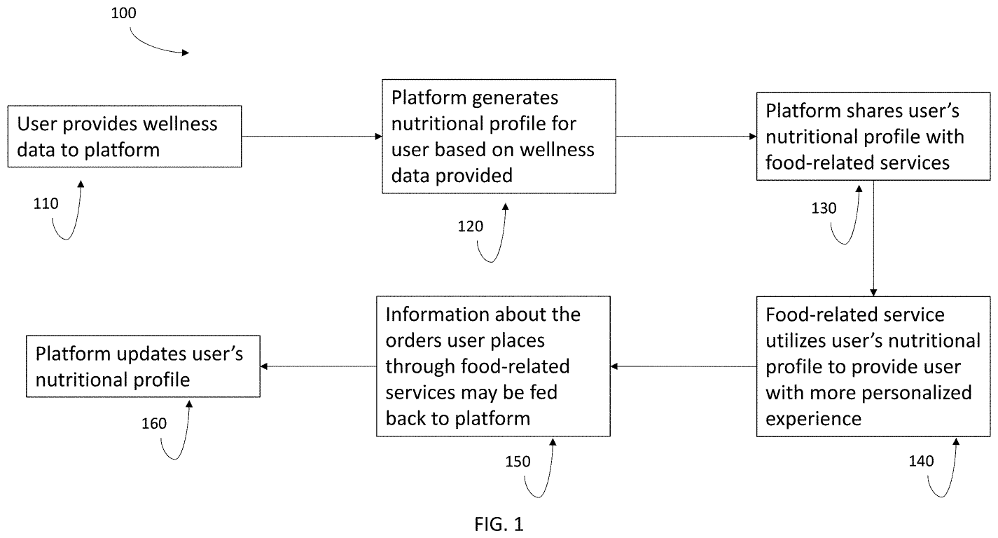

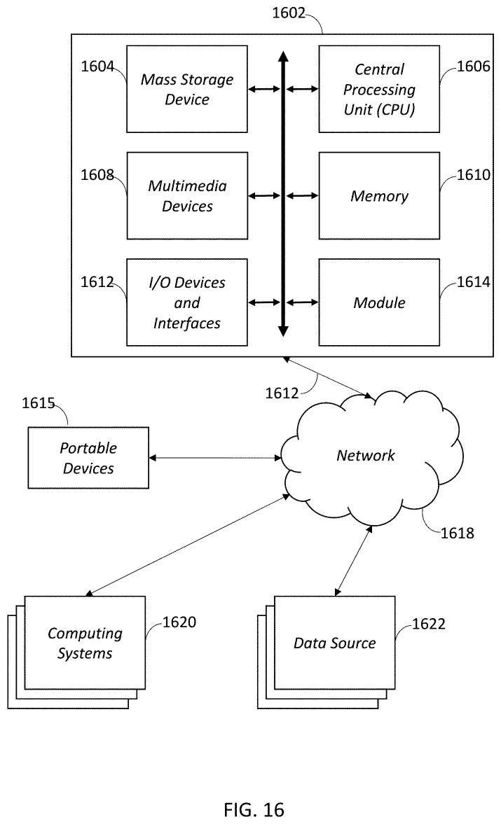

FIG. 1 is a block diagram illustrating an example personalization protocol or method. The method 100 can be implemented, for example, using one or more components of the system shown in FIG. 16.

With reference to FIG. 1, at block 110, a user may provide health or wellness data to a platform. The health or wellness data provided may include results from diagnostic tests, data provided by the user, a wellness professional (e.g., dietician, physician, nutritionist, etc.), one or more health or medical devices, and/or one or more services connected with the platform. In some embodiments, the diagnostic tests may be testing for white blood cell count, kidney function, liver function, hydration levels, pH levels, urinary tract infections, ketones, allergies, etc. The health or wellness data provided may also include data provided by the user, wellness professional (e.g., dietician, physician, nutritionist, etc.), one or more health or medical devices, and/or one or more services connected with the platform. Data provided may be indicative of the user's weight, blood pressure, glucose levels, cholesterol, medical conditions, medications or over the counter products taken by the user, dietary habits and/or preferences, wellness goals, biomarker metrics, etc.

At block 120, the platform may generate a nutritional profile for the user based on the health or wellness data provided. The platform may be a platform that includes remote health and diagnostic testing capabilities.

At block 130, the platform may share the user's nutritional profile with one or more food-related services. Such food-related services may include rapid food delivery services, grocery ordering/delivery services, subscription meal services, independent restaurants/restaurant chains, establishment dining services. For example, rapid food delivery services may include Uber Eats, DoorDash, Grubhub, Seamless, etc. Such grocery ordering or delivery services may include Instacart, Amazon, Whole Foods, Fresh Express, etc. For example, independent restaurants or restaurant chains may include Starbucks, Sweet Green, Chipotle, Caribou, Chick-fil-a, etc. Such establishment dining services may include college dining plans, business cafeterias, etc.

At block 140, the one or more food-related services may utilize the user's nutritional profile to provide the user with a more personalized experience. For example, the personalized experience may include a personalized menu or list of food-related items from which the user can order. The menu or list may cater to the user's nutritional, dietary, and/or wellness needs. In some instances, the personalized experience may include access to foods and/or dishes that are prepared to the user's nutritional, dietary, and/or wellness needs. For examples, individual restaurants may be able to add vitamins and supplements to dishes ordered by the user (e.g., smoothies, pressed juices, shakes, etc.) to address the user's preferences and/or needs. In some instances, the personalized experience may include customized restaurant and/or dish recommendations based on the user's preferences and/or dietary needs or restrictions.

At block 150, information regarding the orders the user may place through the one or more food-related services may be fed back to the platform. At block 160, the platform may update the user's nutritional profiled based on the information received. For example, the information received by the platform may contribute to determining whether a given dietary strategy may be proving to be successful in assisting the user reach its wellness goals, identify possible food allergies, adhere to dietary restrictions, etc.

### Tabletop Labs for Diagnostic Medical Testing

This section relates to tabletop environment (e.g., generally small and/or portable suitable for use at home, remotely, or in lab settings) in which testing, such as diagnostic medical testing, can be administered. A health or diagnostic test may be administered in a controlled environment. As described herein, a tabletop accessory may be provided to the user that allows the user to easily set up an environment to administer the test.

In some embodiments, the user may set up the tabletop accessory. The tabletop accessory may be inexpensive and easy for the user to assemble. The tabletop accessory may include a QR code that directs the user's personal device to a website that includes all of the tests supported by a platform. The user may then scan the QR code and select the test. The user may then administer the test. This can, for example, beneficially provide the user with an environment to administer the health or diagnostic test in.

In some instances, a user may be provided with an accessory that creates an environment to administer testing within. Such testing may include remote diagnostic or health testing. The tabletop accessory provided may be an inexpensive single or multi-use environment. The tabletop accessory may involve complex structures that may be repeatedly collapsible. The tabletop accessory may include a three-dimensional volume that occupies space. The environment may include a light box, phone stand, and/or a back and bottom surface. Such tabletop accessory may be consistent and controlled between all tabletop accessories provided to better ensure the appearance of the environment is recognized by a platform and its algorithms. The tabletop accessory may include at least one QR code, color calibration graphics, platform recognizing identifiers, and/or deliberate fiducials for augmented reality purposes. The QR code may give the user access to at least one test supported by the platform. When scanned, the QR code may direct the user's personal device to a website that provides a list of all the diagnostic tests that may be supported by a platform with accompanying photos of each test. The tabletop accessory may include cardboard and may be disposable as such. Similarly, the tabletop accessory may be flat-packed, which may allow the tabletop accessory to be included inside diagnostic test kits. In this manner, the tabletop accessory may be a stack of cardboard that may be configured to include a folding up mechanism so that the user can unfold the tabletop accessory to set up the environment for testing. The tabletop accessory may further include a flap to insert other testing items if necessary. The stack of cardboard may be similar in size to a gift card, credit card, etc. The tabletop accessory may be structurally sound with the ability to disassemble. The tabletop accessory may connect to a hub or phone dock. The tabletop accessory may further include a diagnostic test kit within the accessory itself and/or a driver's license stand.

In some instances, for example, the tabletop accessory may include a webcam that may be mounted on a top flap of the tabletop accessory. The tabletop accessory may include a socket for the webcam to be exchanged with other sensors. The tabletop accessory may include fiducials that may identify the extrinsic of the tabletop accessory, which may include a serial number for user information. The tabletop accessory may include a camera calibration system that may be built in for optical sensors. The tabletop accessory may further include a bus for other peripherals to be used with the tabletop accessory. Such peripherals may be rentable or purchasable based on an ongoing treatment plan of the user. Such peripherals may include test tubes, microscopes, thermometers, centrifuges, scales, etc.

Ensuring a user has the proper set-up required to administer at home tests may be important. Accordingly, it may be beneficial to provide the user with a tabletop environment in which testing can be administered within. This may allow the user to easily assembly and then administer the health or diagnostic test in an environment that includes items the user may need during administration of the health or diagnostic test. For example, in some cases, the tabletop accessory may include phone stand, lightbox, panorama, or background, and/or a tube holder.

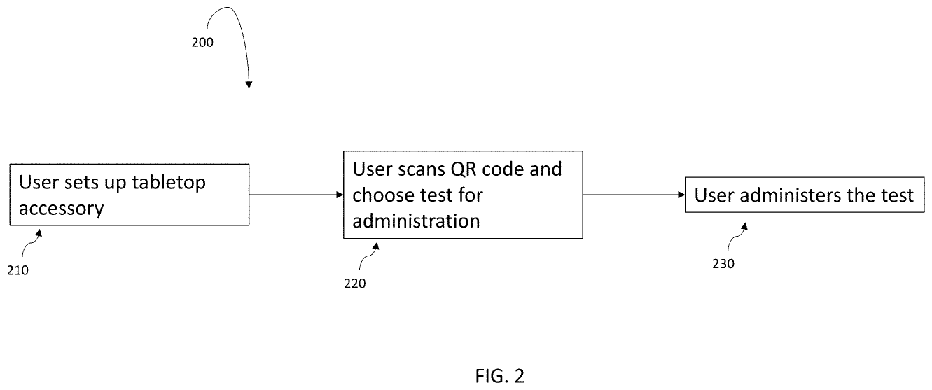

FIG. 2 is a block diagram illustrating an example tabletop accessory protocol or method. The method 200 can be implemented, for example, using one or more components of the system shown in FIG. 2.

At block 210, a user may set up the tabletop accessory. The tabletop accessory may include a lightbox, phone stand, and a back and bottom surface. In some embodiments, the tabletop accessory may be a single or multi use accessory that may be disposable. In some embodiments, the tabletop accessory may be made out of cardboard. In some embodiments, the tabletop accessory may include at least one QR code, color calibration graphic, or other platform recognizable identifiers. In some embodiments, the tabletop accessory may be flat-packed. This may allow the tabletop accessory to include a stack of cardboard that the user may unfold to set up.

At block 220, the user may scan a QR code, which may direct the user's phone to a website that may provide a list of all the diagnostic tests that may be supported by a platform. In some embodiments, the QR code may be scanned for an express/expedited proctoring session. At block 230, the user may administer the test.

In some instances, a user may be provided with an accessory that creates an environment to administer testing within. Such testing may include remote diagnostic or health testing. The tabletop accessory provided may be a multi-use tabletop accessory. The tabletop accessory may be used multiple times. The tabletop accessory may be connected to a user's account, which may maintain at least one credit. Each time the user utilizes the tabletop accessory to administer a test and is issued a result, at least one test credit may be deducted from the user's account.

In some instances, for example, the tabletop accessory may include a card that may be refilled with credits that may be redeemed for proctored testing sessions. The card may be associated with the user's account and may be scanned to display the user's most recent test results. For example, each tabletop accessory may include a plurality of cards, each of which may be used by a different member of the user's family. Similarly, for example, a card may be preloaded with credits and sold in various pharmacies. A platform may issue credits to the user, which may allow the user to use the credits at a future time. The credits may be general credits, which would allow the user to use the credits for any test supported by the platform. Similarly, the credits may not expire. The type of proctoring session or user experience may contribute to the number of credits needed to administer the testing session. For example, a user administered testing session that may be primarily based in artificial intelligence may cost one credit. Similarly, for example, a fully human proctored or verified express or expedited testing session may cost three credits. Furthermore, for example, the type test result issued to the user after the testing session may contribute to the value of the credit.

In some instances, there may be different types of credits for the various types of tests supported by the platform. For example, each tabletop accessory may include one COVID-19 diagnostic test credit, one STREP diagnostic test credit, one FLU test credit, one URINARY TRACT INFECTION diagnostic test credit, etc. This may incentivize users to purchase and administer different tests. In some instances, the cost of the test may be charged to the test manufacturer.

In some instances, for example, the tabletop accessory may involve complex structures that may be repeatedly collapsible. The tabletop accessory may obtain a three-dimensional volume that occupies space. The tabletop accessory may include a light box, phone stand, and/or a back and bottom surface. Such tabletop accessory may be consistent and controlled between all tabletop accessories provided to better ensure the appearance of the environment is recognized by a platform and its algorithms. The tabletop accessory may be flat-packed, which may allow the tabletop accessory to be included inside diagnostic test kits. The tabletop accessory may include at least one QR code, color calibration graphics, platform recognizing identifiers, and/or deliberate fiducials for augmented reality purposes. The QR code may give the user access to at least one test supported by the platform. When scanned, the QR code may direct the user's personal device to a website that provides a list of all the diagnostic tests that may be supported by a platform with accompanying photos of each test. The tabletop accessory may be structurally sound with the ability to disassemble. The tabletop accessory may connect to a hub or phone dock. The tabletop accessory may further include a diagnostic test kit within the accessory itself and/or a driver's license stand.

In some instances, for example, the tabletop accessory may include a webcam that may be mounted on a top flap of the tabletop accessory. The tabletop accessory may include a socket for the webcam to be exchanged with other sensors. The tabletop accessory may include fiducials that may identify the extrinsic of the tabletop accessory, which may include a serial number for user information. The tabletop accessory may include a camera calibration system that may be built in for optical sensors. The tabletop accessory may further include a bus for other peripherals to be used with the tabletop accessory. Such peripherals may be rentable or purchasable based on an ongoing treatment plan of the user. Such peripherals may include test tubes, microscopes, thermometers, centrifuges, scales, etc.

Ensuring the user has the proper set-up required to administer at home tests may be important. Accordingly, it may be beneficial to provide the user with a tabletop environment in which testing can be administered within. This may allow the user to easily assembly and use credits to then administer the health or diagnostic test. For example, in some cases, the user may use at least one credit to begin the administration of the test.

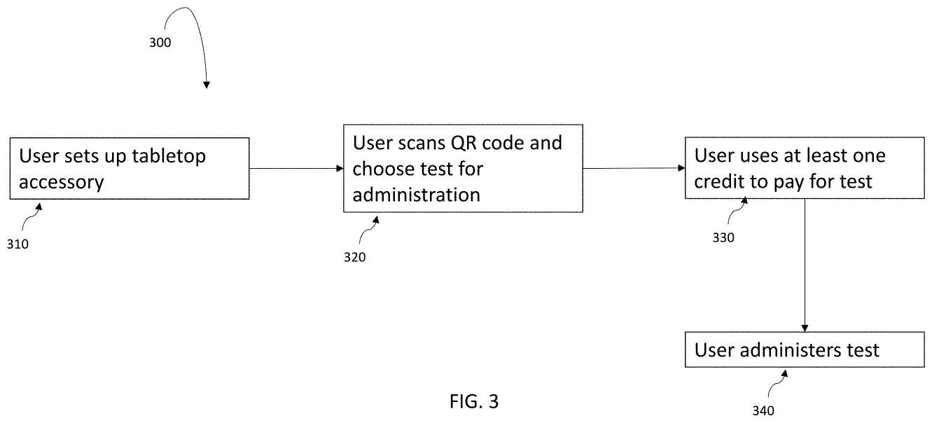

FIG. 3 is a block diagram illustrating an example rechargeable tabletop accessory protocol or method. The method 300 can be implemented, for example, using one or more components of the system shown in FIG. 3.

At block 310, a user may set up the tabletop accessory. The tabletop accessory may include a lightbox, phone stand, and a back and bottom surface. In some embodiments, the tabletop accessory may be a multi-use accessory that may be disposable. In some embodiments, the tabletop accessory may be made out of cardboard. In some embodiments, the tabletop accessory may include at least one QR code, color calibration graphic, or other platform recognizable identifiers. In some embodiments, the tabletop accessory may be flat-packed. This may allow the user to unfold the tabletop accessory for use.

At block 320, the user may scan a QR code, which may direct the user's phone to a website that may provide a list of all the diagnostic tests that may be supported by a platform. The website may allow the user to utilize at least one credit issued to administer the test. In some embodiments, the QR code may be scanned for an express/expedited proctoring session.

At block 330, the use may use at least one credit as payment for the test selected. The credits may be general credits, which would allow the user to use the credits for any test supported by the platform. The type of proctoring session or user experience may contribute to the number of credits needed to administer the testing session. At block 340, the user may administer the test.

### Self-Administered Tests

Remote or at-home medical testing provides significant benefits to patients. However, patients may encounter difficulty acting based on their test results. For example, a patient who takes an at-home Covid-19 test may face difficulty obtaining treatment based on the results of the test, causing the patient to seek out in-person medical testing and treatment, resulting in a worse patient experience and potentially introducing significant delays that may, in some circumstances, have significant impacts on the patient's health. Having to seek out additional in-person testing and treatment can also result in a worse experience for other patients, especially when testing labs and medical providers are operating near or over capacity, such as during surges in Covid-19 cases.

Preferably, patients can obtain treatment quickly based on the results of at-home testing. This can be especially important for some conditions such as Covid-19. For example, oral medications for Covid-19 such as Molnupiravir and co-packaged Nirmatrelvir and Ritonavir tablets need to be taken within a limited timeframe such as, for example, within five days of symptom onset. Similarly, monoclonal antibodies for the treatment of Covid-19 are authorized for use within the first ten days after the onset of symptoms. Thus, it is important to minimize any delays between the onset of symptoms and the start of treatment. Delays may be reduced if patients can obtain treatment through a remote testing platform based on the results of at-home diagnostic testing.

Self-administered treatments may be used for the treatment of certain conditions. For example, multiple oral medications for the treatment of Covid-19 have obtained emergency use authorization. These medications must be prescribed by a healthcare professional (which may be a physician, pharmacist, or other healthcare professional authorized to prescribe treatment), but the patient can take the medication at home. Remote prescription carries several advantages for self-administered treatment. Patients can obtain treatment conveniently at home and without having to go to an in-person provider visit. This is not only advantageous for the patient who can obtain treatment with reduced inconvenience and delay but may also reduce community spread of disease as the patient can stay home instead of traveling to seek medical care. In some cases, medications may be sent directly to the patient's home or may be filled at a pharmacy of the patient's choosing.

In some embodiments, a testing platform may interface with prescribers in a manner that is transparent to the user. For example, the user may take a test, receive a result, and obtain treatment all within the testing platform, with no apparent discontinuity, although a prescribing partner may be a different entity from the provider of the testing platform. In some cases, a third entity may be engaged to facilitate the delivery of treatments to patients, and this too may be done in a manner that is transparent to the user. For example, the user may be able to track prescription fulfillment status, delivery status, and so forth through an application or website offered by the testing platform. In some embodiments, the user may be transferred to third parties for treatment fulfillment. For example, a user who receives a positive Covid-19 test may be directed to a pharmacy that can provide treatment to the user.

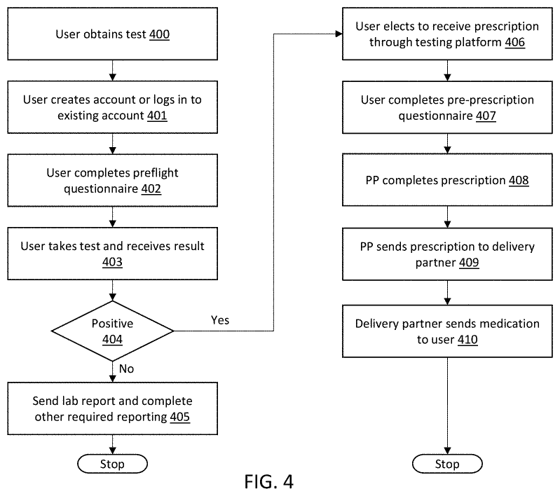

FIG. 4 is an illustration of providing self-administered treatment to a user according to some embodiments. At 400, a user obtains a test. At 401, the user signs into the testing platform, either by creating a new account or by logging into an existing account. At 402, the user completes a pre-flight questionnaire. The pre-flight questionnaire may include, for example, the minimum information required by the testing platform and/or information required to comply with public health requirements. At 403, the user takes the test and receives a result. At 404, if the result is negative, the system may send a lab report and the result and information may be used by the testing platform to complete required reporting at 105. If the test is positive, then at 406 the user may elect to receive a prescription through the testing platform. At 407, the user completes a pre-prescription questionnaire. The pre-prescription questionnaire may include questions related to, for example, the user's medical history, medication contraindications, test results, qualifying criteria (e.g., time since the onset of symptoms), and so forth. In some embodiments, the pre-prescription questionnaire may ask questions related to prescribing a single medication or multiple medications. In some embodiments, the user's answers to the questionnaire may be used to select one treatment from multiple available treatments. At 408, a prescribing partner receives the test results and pre-prescription questionnaire and completes a prescription for the user. At 409, the prescribing partner sends the prescription to a delivery partner which may be, for example, a mail-order pharmacy, a local pharmacy, or, in some circumstances, the prescribing partner (i.e., the prescribing partner may both issue and fulfill the prescription).

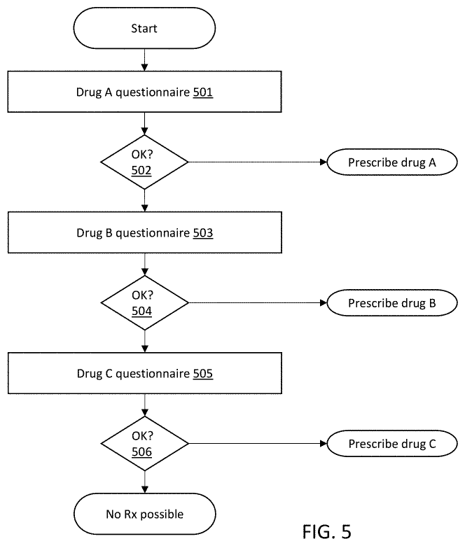

FIG. 5 depicts a drug selection process according to some embodiments which may be implemented on a computer system. At 501, a user completes a questionnaire to determine whether a first drug is an appropriate treatment for the user. For example, the first drug may be an oral medication with a high efficacy rate and the questionnaire may determine whether the user has any conditions or allergies or takes any other medications that would contraindicate the first medication. The questionnaire may ask about health insurance coverage, as not all insurance plans may cover all medications. If, at 502, the system determines that the first drug is okay to prescribe for the user, then the system may proceed with steps for prescribing the first drug, such as sending patient information, test results, and questionnaire responses to a prescribing partner. If, on the other hand, the system determines that the first drug should not be prescribed to the user, the system may present an additional questionnaire for a second drug. The second drug may be, for example, an oral medication with a lower efficacy rate than the first drug. Similarly, the system may present the user with a questionnaire related to a third drug. The third drug may be an injectable, for example, or an oral drug with lesser efficacy than the first drug and the second drug. If none of the available drugs are appropriate for the user, the system may determine that no prescription is possible.

While FIG. 5 illustrates three potential drugs, any number or questionnaires could be presented to screen for any number of medications. In some embodiments, it may be advantageous to combine questions related to different medications into a single questionnaire instead of multiple separate questionnaires. In other embodiments, multiple questionnaires may be used, but responses to earlier questionnaires may be used to avoid asking the user the same questions more than once. For example, if two medications have osteoporosis as a contraindication, there is no need to ask the user about osteoporosis more than once.

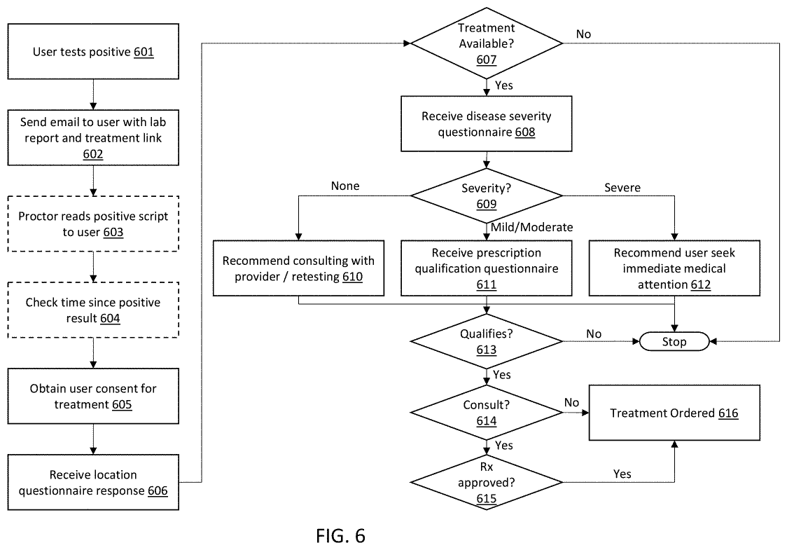

FIG. 6 illustrates a process for prescribing treatment to a user which may be implemented on a computer system. At block 601, a user receives a positive test result, for example by following steps in FIGS. 4-6. In some embodiments, instead of a positive test result, the user may receive a negative test result but have a known exposure that may warrant treatment (e.g., exposure to Covid-19). At block 602, the system sends an email to the user with a report of the results and a link to treatment. The user may use the link if, for example, the user at first declines treatment but later wishes to pursue treatment, or if the user gets disconnected before treatment can be ordered. At block 603, a proctor may optionally read a script to the user indicating a positive test result and advising the user that treatment may be available. At 604, the system may optionally check the amount of time that has elapsed since the user received a positive test result. In some cases, users will proceed to the treatment prescription process immediately after receiving a positive test result. However, in other cases, users may at first decline treatment (for example, if they are not having symptoms at the time) and may return later to seek treatment. If the time since the positive test result is over a threshold value, the user may be advised that a new test is needed.

If the time since testing is below the threshold value, the system may ask the user to consent to treatment at block 605. At block 606, the system may receive a response from the user indicating the user's location. At block 607, the system may determine whether treatment is available based on the user's location. For example, some states may not permit prescribing certain treatments without an in-person visit to a provider. If treatment is available to the user, then at block 608, the system may receive responses to a disease severity questionnaire from the user. If the user has no symptoms, the system may recommend that the user consult with a provider, retest, or wait to see if symptoms appear. If the user has severe symptoms, the system may recommend that the user seek immediate medical attention. If the user has mild or moderate symptoms, the system may provide the user with a prescription qualification questionnaire and may receive responses at block 611. The questionnaire may inquire as to the user's age, medical history, allergies, insurance, and so forth. At block 613, the system may determine if the user qualifies for a prescription. If the user does not qualify, the system may inform the user of the reason or reasons for disqualification and be advised of other steps the user can take. If the user qualifies for a prescription, the system may then determine at block 614 whether the user needs to consult with a prescribing partner such as a provider or pharmacist before treatment can be ordered. For example, some states may require that a user meets with a pharmacist before receiving treatment. In other cases, a user's responses to the prescription qualification questionnaire may warrant further inquiry by a prescribing partner. In some cases, the system may provide a mechanism such as text-based chat or video conferencing to allow the user to consult with a pharmacist or provider. If a consultation is not needed, treatment may be ordered without a consultation. If a consultation is needed, however, then the prescribing partner may approve the prescription and order treatment after consultation with the user or the prescribing partner may determine that treatment is not appropriate for the user.

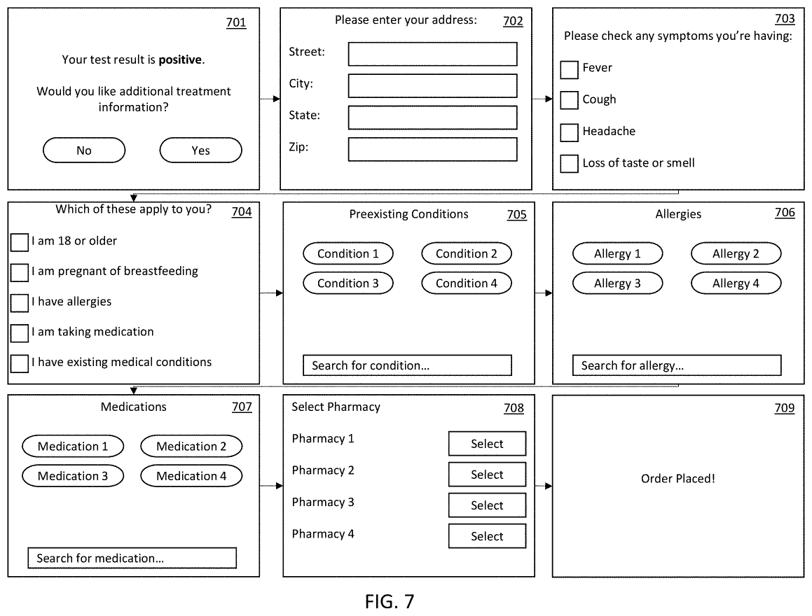

FIG. 7 illustrates a series of screens that may be presented to a user following a positive test result. A computer system may be configured to provide the screens to the user. At screen 701, the system may show the user a screen indicating that the user's test result is positive. The user may be given an option to receive additional treatment information. If the user chooses to receive additional treatment information, the system may show screen 702 to the user, where the user may enter location information which may be used to determine whether the user is eligible for remote treatment. If, for example, the user enters a state where remote prescription of treatment is not permitted, the system may abort the questionnaire and display a message to the user explaining why they are not eligible and advising them of other treatment options. The information supplied by the user on screen 702 may subsequently be used to determine nearby pharmacies in later steps. The system may present the user with screen 703, where the user can select any symptoms that the user is experiencing. If the system determines that the user is experiencing symptoms consistent with remote treatment (for example, mild or moderate symptoms of Covid-19), then the system may then display screens 704 through 707 to the user to determine if the user is eligible for one or more treatments. If the user indicates that they are not experiencing systems, the system may abort the questionnaire and explain that the user is not currently eligible for treatment. If the user indicates that they are having severe symptoms, the questionnaire may abort and advise the user to seek immediate medical attention.

In some embodiments, certain screens may be skipped based on the responses at screen 704. For example, if the user indicates that are not taking any medications, then screen 707 may be skipped because there is no need to inquire about medications if it has already been ascertained that the user is not taking any medications. At any point, the system may abort the questionnaire if the user's responses indicate that the user is not eligible for treatment. If the user is eligible, the user may be prompted to select a pharmacy at screen 708. In some embodiments, the screen 708 may be prefilled with pharmacies that are near the address entered by the patient at screen 702, or the patient may input a zip code, street address, or the like to locate nearby pharmacies. In some cases, before selecting a pharmacy or after selecting a pharmacy, or at some other point, the user may be prompted to consult with a prescribing partner such as a physician or pharmacist. Once the required information is collected and the order is approved by a prescribing partner, the system may display to the user a screen 709 indicating that the order has been placed. In some embodiments, the system may, instead of sending an order to a user's local pharmacy, send the order to a mail-order pharmacy or the like. In some embodiments, the system may be further configured to display an order status, shipment status, shipment tracking number, or the like.

Unlike self-administered treatments such as oral medications, some treatments must be professionally administered. Monoclonal antibodies, for example, may be used for the treatment of Covid-19. Monoclonal antibodies are delivered as an infusion and must be professionally administered at a treatment location such as a hospital or clinic. Thus, even if a patient takes an at-home test, the patient will still have to travel to obtain treatment. Even in such cases, however, it may be advantageous for the patient to obtain a drug order for monoclonal antibodies through a remote testing platform as the patient can avoid having to visit a medical facility to determine whether monoclonal antibodies are an appropriate treatment and to obtain an order for the monoclonal antibodies, potentially saving time and allowing for earlier treatment.

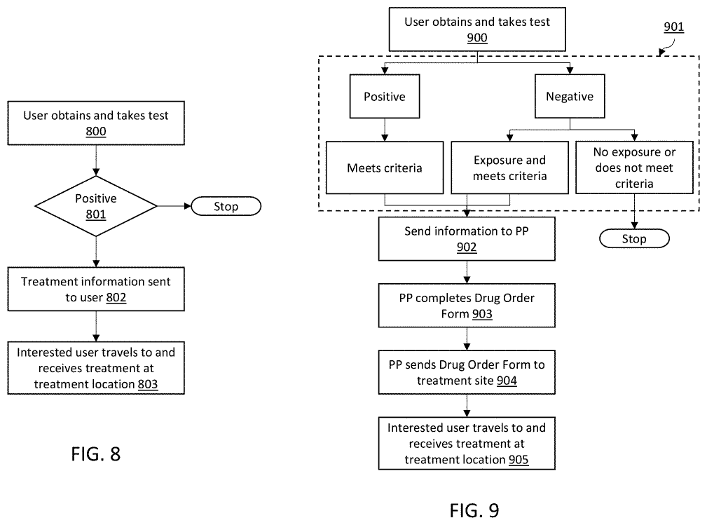

FIG. 8 shows an illustration of professionally-administered treatment according to some embodiments. At block 800, a user obtains and takes a diagnostic test, such as, for example, a Covid-19 test. At block 801, the result is negative, the user is finished with the process and no further action is taken. If, however, at block 801, the test result is positive, a testing platform may send the user information about professionally-administered treatment (e.g., monoclonal antibody treatment). The information provided to the patient may include treatment sites, information about eligibility, and so forth. At block 803, an interested user travels to a treatment location and receives treatment if a provider determines that treatment is appropriate. Under the process depicted in FIG. 8, determining whether the user is eligible for treatment and placing orders for treatment are outside the scope of the remote testing and treatment process. Instead, a user is provided with information that helps them locate treatment sites and providers who can order treatment.

FIG. 6 shows an illustration of professionally-administered treatment according to some embodiments. At block 900, a user obtains and takes a diagnostic test such as, for example, a Covid-19 test. At block 901, the patient may test positive and meet the criteria for treatment, may test negative and have been exposed and meet the criteria for treatment, or may test negative and either not have a known exposure, not meet the treatment criteria, or both. If a user meets the treatment criteria and either tests positive or tests negative but has a known exposure, the patient's personal information, test results, and other information such as, for example, any questionnaires or the like that the user completed may, at block 902, be sent to a prescription partner (PP) for review. The prescription partner, upon determining that treatment is appropriate for the user, may at block 903 complete a Drug Order Form (for example, for monoclonal antibody treatment) and then, at block 904, send the Drug Order Form to a treatment site. At block 905, an interested user travels to the treatment site and receives treatment.

### Rapid Tests

While remote and at-home testing offer several advantages, there may be significant limitations. For example, users of remote or at-home testing may be more limited than the testing options available at a medical provider or commercial laboratory. For example, commonly available at-home tests for COVID-19 are antigen tests that look for fragments of proteins from the SARS-CoV-2 virus in order to determine if a person has an active infection. While antigen testing is useful for quickly determining if someone has an active infection, antigen tests are less sensitive than molecular tests such as loop-mediated isothermal amplification (LAMP) and may yield false negative results. LAMP testing may be more sensitive than antigen testing, and thus may be used to determine with greater reliably if someone did not have an active COVID-19 infection at the time of testing.

Preferably, at-home testing users, users in remote locations without access to laboratory testing, and even medical providers could access molecular testing such as LAMP testing without the need to engage with a laboratory or to purchase laboratory equipment which may be expensive, require reliable electricity, and require specialized knowledge to operate. The embodiments disclosed herein could be used by remote healthcare providers, individual providers or clinics, or individual users to perform rapid molecular testing with relative ease and at relatively low cost.

Molecular testing may be used in place of or in addition to other testing methods such as, for example, antibody testing and antigen testing, to screen for various conditions. For example, molecular testing may be used to screen for Zika, tuberculosis, malaria, SARS-CoV-2, and so forth. Various molecular testing techniques may be used such as polymerase chain reaction and loop-mediated isothermal amplification.

For testing at home, in remote regions, and in clinics that do not have well-equipped laboratories, LAMP may offer several advantages. In contrast to PCR, which operates at various temperatures throughout the process, LAMP is isothermal, eliminating the need for controlled thermal cycling. Moreover, LAMP results may be interpreted by visual inspection such as looking for a change in the color or translucency of the solution.

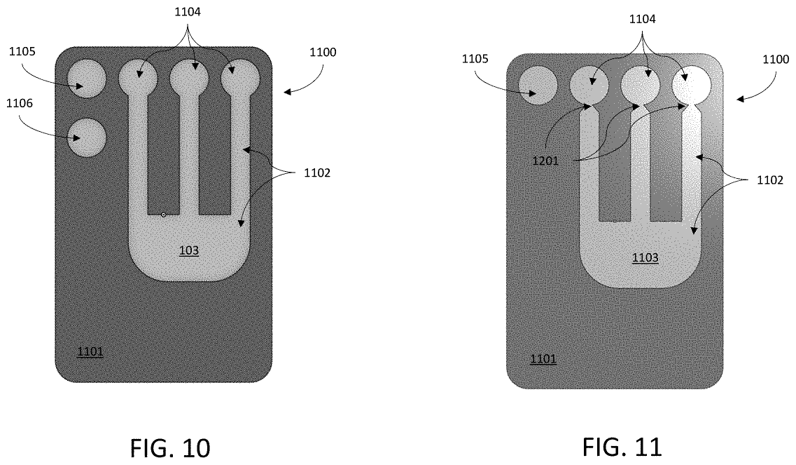

FIG. 10 depicts a test card according to some embodiments which may be used for LAMP testing or other molecular testing. The test card 1100 has a hydrophobic region 1101, a hydrophilic region 1102, a sample well 1103, test wells 1104, and optionally, a high temperature indicator 1105 and a low temperature indicator 1106. The hydrophobic region 1101 may block wicking of fluid to keep the fluid in desired portions of the test card. The hydrophobic region 1101 is preferably non-reactive such that it does not reaction with the sample fluid or the hydrophilic region 1102. The hydrophilic region 1102 may be non-reactive such that it does not react with sample fluid or reagents and does not bind to biological molecules such as, for example, DNA or RNA. Preferably, the hydrophilic region 1102 provides a flow rate sufficient to distribute sample fluid quickly. The hydrophilic region 1102 may advantageously tolerate a range of temperatures such that the card remains functional at a variety of storage, transport, and reaction temperatures. The sample well 1103 may receive saliva or another solution containing a sample to be measured. The test wells 1104 may comprise control wells, target wells, or both. For example, a control well may be configured to test for something that exists in human saliva. The test well may be used to determine that the correct conditions exist for loop-mediated isothermal amplification. Target wells may be used to carry out reactions. For example, a test card 1100 may be configured with two target wells to test for two different viruses of interest. In some embodiments, the test card 1100 may be extended horizontally to accommodate additional test wells which may act as control wells, target wells, or both. Optionally, the test card 1100 may be configured with a high temperature indicator 1105. The high temperature indicator 1105 may change color permanently if the test card exceeds a maximum allowable temperature for more than a threshold time. Similarly, a test card may be configured with a minimum temperature indicator 1106 that permanently changes color upon being exposed to a temperature that is below a minimum temperature for more than a threshold time.

The test card depicted in FIG. 10 may be modified or enhanced with additional features. For example, as shown in FIG. 11, the flow regions between the sample well 1103 and the test wells 1104 may be narrowed near the test wells 1104 to form bottlenecks 1201 that may be used to slow the exchange of fluid and mitigate mixing and flow that may occur outside the test wells.

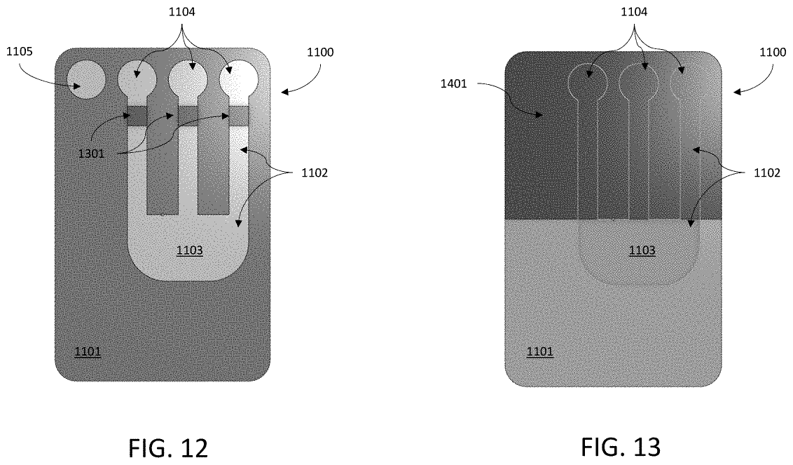

In some embodiments, wax valve seals may be used as shown in FIG. 12. For example, wax valve seals 1301 may be placed near the test wells and may melt so as to seal off the test wells when reactions begin or when heating is applied to the test card 100, thereby preventing flow out of the test wells 1104.

As shown in FIG. 13, in some embodiments, a sticker 1401 or other cover may be placed over the flow regions and the test wells 1104 to prevent users from touching the test wells. In some embodiments, the sticker 1401 may be transparent so that the user can observe a change in color, translucency, and so forth in the test wells 1104.

Test cards may be prepared from a variety of materials. Generally, test cards comprise a hydrophobic base with a section that can receive a hydrophilic material. For example, a hydrophobic base may be made of plastic or cardboard. An injection-molded plastic, for example, may serve as a structural component, or may be a thin part that can be attached to a cardboard back to provide rigidity. In some embodiments, the hydrophobic base may be constructed from cardboard. For example, a piece of cardboard with a cutout for a hydrophilic region may be affixed (e.g., by gluing) to a flat piece of cardboard. in some embodiments, a single piece of cardboard may be used, and an indentation for a hydrophilic region may be formed by, for example, stamping the cardboard. Cardboard may be made hydrophobic by, for example, wax impregnation, wax or plastic dipping, spraying with acrylic or another sealant, plastic thermoforming, and so forth. The hydrophilic material may be, for example, a common lateral flow substrate such as nitrocellulose. However, in some embodiments, typical lateral flow substrates may have unacceptable biomolecule binding. Thus, in some embodiments, a different material such as polyethersulfone may be used. The hydrophilic material may be shaped by, for example, die cutting or laser cutting. Die cutting may be preferable in some use cases because it is easy, accurate, and low cost. However, in some cases, such as, for example, when a limited number of test cards are needed, laser cutting or other cutting methods may be preferable.

In some embodiments, a test card may be made up of a single material. For example, a test card may start as a single hydrophilic slab, and hydrophobic regions may be formed by treating the hydrophilic material. For example, hydrophobic regions may be formed using photolithography, wax printing, or inkjet printing with a chemical that imparts hydrophobic properties. In some embodiments, stereolithography may be used to form a hydrophobic region. For example, the material may be soaked in an ultraviolet cure resin and a UV laser may be used to selectively cure a hydrophobic region, and the uncured resin may be washed away. In some embodiments, a wax vacuum approach may be used by soaking the material in molten wax and then applying a vacuum selectively to the hydrophilic region to remove the molten wax.

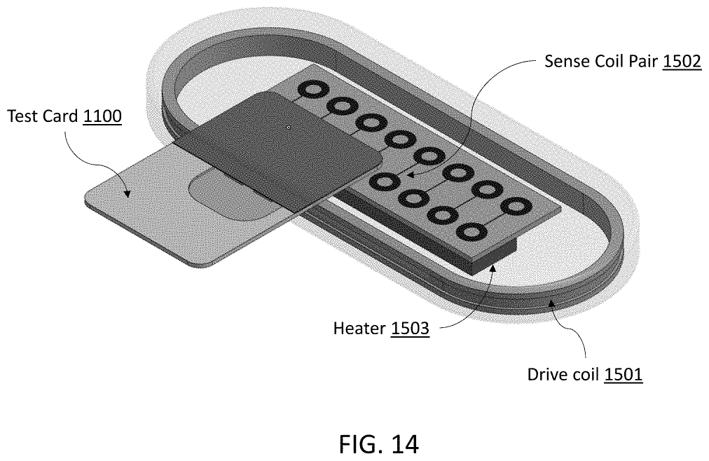

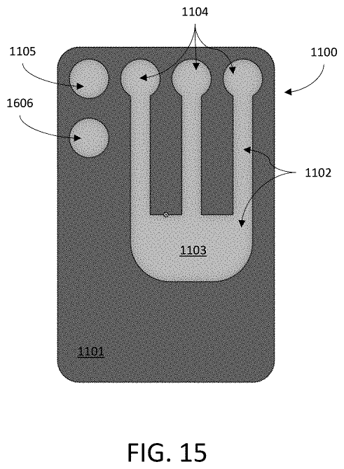

FIG. 14 is an illustration of a reader that may be used to read test cards, such as the test card 1100, according to some embodiments. The reader 1500 comprises a drive coil 1501, sense coil pairs 1502, and a heater 1503. The reader 1500 may be configured to accept one two card, two test cards, or more test cards.

In some cases, it may be advantageous to be able to perform testing without access to an external electricity source. The example, there may be a need to perform testing in areas that have lost electricity due to a natural disaster or in remote regions that lack access to reliable electricity. Battery power can be used, but this can lead to environmental damage due to the need to produce single-use batteries and the potential that single-use batteries will not be disposed of properly. Thus, there is a need for a PCR test that can work without access to electricity and preferably without the use of single-use batteries.

However, it can be important that a LAMP reaction takes place in a limited temperature range such as, for example, from about 55° C. to about 95° C. LAMP reactions used for diagnostic testing may be stable within a range of about 10° C. In some embodiments, an exothermic reaction may be used to provide a heat source for LAMP reactions. For example, an iron oxidation reaction, such as that used by disposable hand warmers in sodium chloride is used to catalyze the reaction between iron filings and air, may be used to provide a heat source of suitable temperature and stability for the LAMP reaction. The iron oxide reaction is non-toxic but is also non-reversible, so a new heat source would be needed for each new testing session.

Other exothermic reactions may be used such as, for example, nucleation and crystallization on a metal surface that is embedded in a supersaturated sodium acetate solution. Mechanical flexing may be used to trigger the reaction, and the reaction may be reversed by heating to dissolve any formed crystals back into the solution, allowing the same heat source to be reused for multiple testing sessions.

In some embodiments, the exothermic reaction may be used as a direct heating source for the LAMP reaction. In some other embodiments, the exothermic reaction may be used indirectly. For example, the exothermic reaction may be used in conjunction with a thermoelectric generator such as a Peltier heat pump to provide power for heating and other functionality.

In some embodiments, a test card such as the test card 1100 depicted in FIGS. 1 through 4 may be modified to enable higher reliability of test results obtained by testing that uses exothermic reactions to provide heating. For example, as shown in FIG. 15, the test card 1100 may include a minimum temperature indicator 1606. The minimum temperature indicator 1606 may be configured to change color permanently upon the temperature of the card rising to a minimum temperature for viable LAMP testing. For example, if the minimum temperature indicator 1606 does not change color during testing, the user will know that that the test may not have reached the minimum temperature for the LAMP reaction and thus should not be considered accurate.

Advantageously, LAMP results may be interpreted without the use of optical or electronic sensors. For example, results may be determined by observing changes in the turbidity of the reaction solution. In some cases, intercalating dyes may be used and may produce a color change that is visible to the naked eye. Other visual detection methods may also be used, such as using functionalized gold nanoparticles.

### Computer Systems

FIG. 16 is a block diagram depicting an embodiment of a computer hardware system configured to run software for implementing one or more embodiments of the health testing and diagnostic systems, methods, and devices disclosed herein.

In some embodiments, the systems, processes, and methods described herein are implemented using a computing system, such as the one illustrated in FIG. 16. The example computer system 1602 is in communication with one or more computing systems 1620 and/or one or more data sources 1622 via one or more networks 1618. While FIG. 16 illustrates an embodiment of a computing system 1602, it is recognized that the functionality provided for in the components and modules of computer system 1602 may be combined into fewer components and modules, or further separated into additional components and modules.

The computer system 1602 can comprise a module 1614 that carries out the functions, methods, acts, and/or processes described herein (such as those that allow for customization or personalization of a dietary profile based on user health or wellness data). The module 1614 is executed on the computer system 1602 by a central processing unit 1606 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1602 includes one or more processing units (CPU) 1606, which may comprise a microprocessor. The computer system 1602 further includes a physical memory 1610, such as random-access memory (RAM) for temporary storage of information, a read only memory (ROM) for permanent storage of information, and a mass storage device 1604, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1602 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1602 includes one or more input/output (I/O) devices and interfaces 1612, such as a keyboard, mouse, touch pad, and printer. The I/O devices and interfaces 1612 can include one or more display devices, such as a monitor, that allows the visual presentation of data to a user. More particularly, a display device provides for the presentation of GUIs as application software data, and multi-media presentations, for example. The I/O devices and interfaces 1612 can also provide a communications interface to various external devices. The computer system 1602 may comprise one or more multi-media devices 208, such as speakers, video cards, graphics accelerators, and microphones, for example.

The computer system 1602 may run on a variety of computing devices, such as a server, a Windows server, a Structure Query Language server, a Unix Server, a personal computer, a laptop computer, and so forth. In other embodiments, the computer system 202 may run on a cluster computer system, a mainframe computer system and/or other computing system suitable for controlling and/or communicating with large databases, performing high volume transaction processing, and generating reports from large databases. The computing system 202 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 1602 illustrated in FIG. 16 is coupled to a network 1618, such as a LAN, WAN, or the Internet via a communication link 1616 (wired, wireless, or a combination thereof). Network 1618 communicates with various computing devices and/or other electronic devices. Network 1618 is communicating with one or more computing systems 1620 and one or more data sources 1622. The module 1614 may access or may be accessed by computing systems 1620 and/or data sources 1622 through a web-enabled user access point. Connections may be a direct physical connection, a virtual connection, and other connection type. The web-enabled user access point may comprise a browser module that uses text, graphics, audio, video, and other media to present data and to allow interaction with data via the network 1618.

Access to the module 1614 of the computer system 1602 by computing systems 1620 and/or by data sources 1622 may be through a web-enabled user access point such as the computing systems' 1620 or data source's 1622 personal computer, cellular phone, smartphone, laptop, tablet computer, e-reader device, audio player, or another device capable of connecting to the network 1618. Such a device may have a browser module that is implemented as a module that uses text, graphics, audio, video, and other media to present data and to allow interaction with data via the network 1618.

The output module may be implemented as a combination of an all-points addressable display such as a cathode ray tube (CRT), a liquid crystal display (LCD), a plasma display, or other types and/or combinations of displays. The output module may be implemented to communicate with input devices 1612 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1602 may comprise a physical or logical connection established between a remote microprocessor and a mainframe host computer for the express purpose of uploading, downloading, or viewing interactive data and databases online in real time. The remote microprocessor may be operated by an entity operating the computer system 1602, including the client server systems or the main server system, an/or may be operated by one or more of the data sources 1622 and/or one or more of the computing systems 1620. In some embodiments, terminal emulation software may be used on the microprocessor for participating in the micro-mainframe link.

In some embodiments, computing systems 1620 who are internal to an entity operating the computer system 1602 may access the test integrity module 1614 internally as an application or process run by the CPU 1606.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 1602 may include one or more internal and/or external data sources (for example, data sources 1622). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 1602 may also access one or more databases 1622. The databases 1622 may be stored in a database or data repository. The computer system 1602 may access the one or more databases 1622 through a network 1618 or may directly access the database or data repository through I/O devices and interfaces 1612. The data repository storing the one or more databases 1622 may reside within the computer system 1602.

### Other Embodiments

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein, but are to be accorded the widest scope consistent with this disclosure, the principles and the novel features disclosed herein.

What is claimed is:

1. A computer-implemented method, comprising:
receiving, by a computer system, patient information from a patient in communication with the computer system over an electronic network, the patient information comprising a result of at least one remotely-administered diagnostic test, wherein the at least one remotely-administered diagnostic test is facilitated through a testing platform accessed by the patient over the electronic network;
generating, by the computer system, a nutritional profile for the patient based on the patient information;
transmitting, by the computer system, the nutritional profile over the electronic network to one or more food-related services, wherein the food-related services provide a service to the user based at least in part on the nutritional profile;
receiving, by the computer system, information regarding an interaction between the patient and at least one of the food-related services, wherein the information is sent by the food-related services over the electronic network to the computer system; and
updating, by the computer system, the nutritional profile of the patient based on the information received from the at least one of the food-related services.

2. The method of claim 1, wherein the patient information comprises one or more of:
a white blood cell count of the patient;
an indicator of kidney function for the patient;
an indicator of liver function for the patient;
an indication of a hydration level for the patient;
an indicator of a pH level of the patient;
an indicator of a measure of ketones of the patient; and/or
allergy information for the patient.

3. The method of claim 1, further comprising:
receiving, by a computer system, additional patient information from a healthcare provider or medical device in communication with the computer system over an electronic network, the patient information comprising a result of at least one remotely-administered diagnostic test, wherein the at least one remotely-administered diagnostic test is facilitated through a testing platform accessed by the patient over the electronic network.

4. The method of claim 3, wherein the additional information comprises one or more of:
a weight of the patient;
a blood pressure of the patient;
a glucose level of the patient;
a cholesterol measure of the patient;
a medical condition of the patient;
a medications taken by the patient;
dietary habits and/or preferences of the patient;
a wellness goal of the patient; and/or
a biomarker metric of the patient.

5. The method of claim 3, wherein the nutritional profile is further based on the additional information.

6. The method of claim 1, wherein the food-related services comprises one or more of the following:
a rapid food delivery service;
a grocery ordering and/or delivery service;
a subscription meal services;
a restaurant; and/or
an establishment dining service.

7. A system comprising at least one memory storing instructions that cause at least one processor to:
receive patient information from a patient in communication with the computer system over an electronic network, the patient information comprising a result of at least one remotely-administered diagnostic test, wherein the at least one remotely-administered diagnostic test is facilitated through a testing platform accessed by the patient over the electronic network;
generate a nutritional profile for the patient based on the patient information;
transmit the nutritional profile over the electronic network to one or more food-related services, wherein the food-related services provide a service to the user based at least in part on the nutritional profile;
receive information regarding an interaction between the patient and at least one of the food-related services, wherein the information is sent by the food-related services over the electronic network to the computer system; and
update the nutritional profile of the patient based on the information received from the at least one of the food-related services.

8. The system of claim 8, wherein the patient information comprises one or more of:
a white blood cell count of the patient;
an indicator of kidney function for the patient;
an indicator of liver function for the patient;
an indication of a hydration level for the patient;
an indicator of a pH level of the patient;
an indicator of a measure of ketones of the patient; and/or
allergy information for the patient.

9. The system of claim 8, wherein the instructions further cause the processor to:
receive additional patient information from a healthcare provider or medical device in communication with the computer system over an electronic network, the patient information comprising a result of at least one remotely-administered diagnostic test, wherein the at least one remotely-administered diagnostic test is facilitated through a testing platform accessed by the patient over the electronic network.

10. The system of claim 9, wherein the additional information comprises one or more of:
a weight of the patient;
a blood pressure of the patient;
a glucose level of the patient;
a cholesterol measure of the patient;
a medical condition of the patient;
a medications taken by the patient;
dietary habits and/or preferences of the patient;
a wellness goal of the patient; and/or
a biomarker metric of the patient.

11. The system of claim 9, wherein the nutritional profile is further based on the additional information.

12. The system of claim 8, wherein the food-related services comprises one or more of the following:
a rapid food delivery service;
a grocery ordering and/or delivery service;
a subscription meal services;
a restaurant; and/or
an establishment dining service.

13. A computer-implemented method, comprising:
determining, by a computer system, a test result of a user, wherein the test result based on a self-administered diagnostic medical test;
based upon a determination that the test result is positive, wherein a positive test result indicates that treatment is advisable:
transmitting, by the computer system, a prescription questionnaire to the user, the prescription questionnaire, wherein the prescription questionnaire is transmitted to the user over an electronic network and displayed to the user on a user device;
receiving, by the computer system, user inputs in response to the prescription questionnaire, wherein the user inputs are inputted by the user on the user device and transmitted to the computer system over the electronic network;
transmitting, by the computer system, the user inputs and the test result to a prescription provider, wherein the user inputs are transmitted over the electronic network and displayed the prescription provider on a provider device, and wherein the prescription provider determines at least one prescription for the user based on the user inputs and the test result;
receiving, by the computer system, prescription information based on the at least one prescription determined by the prescription provider, wherein the prescription information is transmitted to the computer system over the electronic network;
determining, by the computer system, a delivery partner based on a location of the user and a location of the prescription provider, a delivery partner for delivering the prescription to the user; and
generating, by the computer system, delivery instructions for the delivery partner, wherein the delivery instructions are configured to facilitate the delivery partner obtaining the prescription from the prescription provider and delivering the prescription to the user; and
transmitting, by the computer system, the delivery instructions the delivery partner, wherein the delivery instructions are transmitted over the electronic network.

14. The method of claim 13, wherein the self-administered medical diagnostic test is taken by the user using a testing platform provided by the computer system that provides a video conference connection between the user and a proctor.

15. The method of claim 13, further comprising determining, by the computer system, the prescription questionnaire based on the test result and a database of available medications.

16. The method of claim 13, wherein the prescription questionnaire is further based on a user profile, wherein the user profile is generated based on an interaction between the user and using a testing platform provided by the computer system that facilitates the self-administered medical diagnostic test.

17. The method of claim 13, further comprising determining, by the computer system, if more than a threshold period of time has passed since the user obtained the valid positive test result.
